SEQUENCE LISTING (33 sequences)

SEQ ID NO: 1 - DNA, 21 bp, Artificial sequence
  chemically synthetized
aaggaggcgcccaagcgcccg

SEQ ID NO: 2 - DNA, 23 bp, artificial sequence
  chemically synthetized
aattaaccctcactaaagggaga

SEQ ID NO: 3 - DNA, 21 bp, artificial sequence
  chemically synthetized
tggggtacaggctggcaaagt

SEQ ID NO: 4 - DNA, 16 bp, artificial sequence
  chemically synthetized
gtccttgaccccactt

SEQ ID NO: 5 - DNA, 17 bp, artificial sequence
  chemically synthetized
aggataaggacgacgac

SEQ ID NO: 6 - DNA, 20 bp, Artificial sequence
  chemically synthetized
ccattttctcgccgatttta

SEQ ID NO: 7 - DNA, 20 bp, artificial sequence
  chemically synthetized
gccgcatttgaacgttttat

SEQ ID NO: 8 - DNA, 17 bp, artificial sequence
  chemically synthetized
acctcgtacgctttttg

SEQ ID NO: 9 - DNA, 20 bp, artificial sequence
  chemically synthethized
gtacaacagcgtgtcgtgct

SEQ ID NO: 10 - DNA, 20 bp, artificial sequence
  chemically synthetized
caccggccatcaagtttatc

SEQ ID NO: 11 - DNA, 19 bp, artificial sequence
  chemically synthetized
gtagaagttcttcagctgc

SEQ ID NO: 12 - DNA, 19 bp, artificial sequence
  chemically synthetized
cgtagatgactcgaagctg

SEQ ID NO: 13 - DNA, 18 bp, artificial sequence
  chemically synthetized
accatcctcgacaagcag

SEQ ID NO: 14 - DNA, 18 bp, artificial sequence
  chemically synthetized
gagaggcagggaaagagg

SEQ ID NO: 15 - DNA, 20 bp, artificial sequence
  chemically synthetized
ctcgatcgaatccgtaaaca

SEQ ID NO: 16 - DNA, 21 bp, artificial sequence
  chemically synthetized
ccgcgtatgttttatcgagac

SEQ ID NO: 17 - DNA, 17 bp, artificial
  chemically synthetized
atccttggaccgagcta

SEQ ID NO: 18 - DNA, 17 bp, artificial sequence
  chemically synthetized
atccttggaccgaactc

SEQ ID NO: 19 - DNA, 20 bp, artificial sequence
  chemically synthetized
aggtccaacatgcacagtga

SEQ ID NO: 20 - DNA, 20 bp, artificial sequence
  chemically synthetized
ggcaaagaaaatgtgggcta

SEQ ID NO: 21 - DNA, 17 bp, artificial sequence
  chemically synthetized
ggatacaaactttggaa

SEQ ID NO: 22 - DNA, 20 bp, artificial sequence
  chemically synthetized
agcgaattcgagatgctgnt

SEQ ID NO: 23 - DNA, 20 bp, artificial sequence
  chemically synthetized
ccttnatcttgctgcgcttc

SEQ ID NO: 24 - DNA, 16 bp, artificial sequence
  chemically synthetized
caagctgacggacatt

SEQ ID NO: 25 - DNA, 16 bp, artificial sequence
  chemically synthetized
caagctgacggagatc

SEQ ID NO: 26 - DNA, 20 bp, artificial sequence
  chemically synthetized
ggtacaacatcatcaacttc

SEQ ID NO: 27 - DNA, 16 bp, artificial sequence
  chemically synthetized
gggacataggccagag

SEQ ID NO: 28 - DNA, 20 bp, artificial sequence
  chemically synthetized
agaatccgtatctccatata

SEQ ID NO: 29 - DNA, 20 bp, artificial
  chemically synthetized
cagaatccgtatctccatat

SEQ ID NO: 30 - DNA, 18 bp, artificial sequence
  chemically synthetized
gttgatacgcatgttttt

SEQ ID NO: 31 - DNA, 15 bp, artificial sequence
  chemically synthetized
gcacgtgcacttgat

SEQ ID NO: 32 - DNA, 20 bp, artificial sequence
  chemically synthetized
cagtaaatactgtcggtctc

SEQ ID NO: 33 - DNA, 20 bp, artificial sequence
  chemically synthetized
ccagggtactataacacaga

(19) United States
(12) Patent Application Publication
(10) Pub. No.: US 2008/0241818 A1
(43) Pub. Date: Oct. 2, 2008

(54) Method and microarray for detecting herpesviruses

(75) Inventors: Maija Lappalainen, Espoo (FI); Antti Vaheri, Helsinki (FI); Anne Jaaskelainen, Porvoo (FI); Heli Piiparinen, Sipoo (FI)

Correspondence Address: DODDS & ASSOCIATES, 1707 N STREET NW, WASHINGTON, DC, 20036, US

(73) Assignee: Licentia Ltd., Helsinki (FI)

(21) Appl. No.: 11/879,983

(22) Filed: Jul. 19, 2007

Related U.S. Application Data
(63) Continuation-in-part of application No. PCT/FI07/50182, filed on Apr. 2, 2007.

(51) Int. Cl. C12Q 1/70; C07H 21/00; C40B 40/06; C40B 50/14

(52) U.S. Cl. 435/5; 506/16; 506/30; 536/23.1; 536/24.33

(57) ABSTRACT

The present invention relates to a method and to a microarray for detecting herpesviruses. The invention provides new primers and oligonucleotides for detecting herpesviruses, in particular herpesviruses selected from the group comprising HSV-1, HSV-2, CMV, EBV, VZV, HHV-6A, HHV-6B and HHV-7. By using the method of the invention several different herpesviruses can be detected simultaneously from the same biological sample.

### PRIORITY

This application is a Continuation in Part application of the International Patent Application number PCT/FI2007/050182 which designates United States. The International patent application is incorporated herein by reference. This International application claims priority of U.S. Provisional application No. 60/788,307 filed on Mar. 31, 2006 and which is also incorporated herein by reference.

### SEQUENCE DATA

This application contains sequence data provided on a computer readable diskette and as a paper version. The paper version of the sequence data is identical to the data provided on the diskette.

### METHOD AND MICROARRAY FOR DETECTING HERPESVIRUSES

The present invention relates to a method and to a microarray for detecting herpesviruses. Herpesviruses cause a number of human diseases of clinical importance. Especially diseases of the central nervous system (CNS) and complications in immunocomprimised patients can be the result of infection or reactivation by herpes simplex type 1 or 2 (HSV-1, HSV-2), cytomegalo- (CMV), Epstein-Barr (EBV), varicella zoster (VZV) or human herpes virus 6 (HHV-6) (Aurelius et al., 1991; Koskiniemi et al., 2002; Piiparinen et al. 2002, Aalto et al., 2003). In addition to HHV-6, human herpes virus 7 (HHV-7) has been reported to be a causative agent of exanthema subitum and documented to cause severe complications in transplant recipients (Tanaka et al., 1994; Suga et al., 1997). Virus isolation, nucleic acid detection and a variety of serological methods are currently used to detect herpesvirus infections (Chiu et al., 1998; Pitkaranta et al., 2000; Espy et al., 2000; Read et al., 2001; Ihira et al., 2002). Multiplex-PCR assays have been developed in order to meet the demand for fast detection of herpes viruses (Read and Kurtz, 1999; Aberle and Puchhammer-Stöckl, 2002; Druce et al., 2002; Hudnall et al., 2004). A single tube PCR-assay for simultaneous amplification of various herpes viruses is disclosed in Yamamoto and Nakamura (2000) and differentiation and quantitation of human herpesviruses by real-time PCR in Safronetz et al. (2003). US 2004/0110195 discloses a method for detecting and typing human herpesviruses involving the use of multiplex PCR assays and consensus primers to amplify conserved regions of herpesvirus DNA. US 20040053264 discloses a method for simultaneous detection of pathogenic organisms including herpesviruses by using a DNA chip. US 20030228599 discloses a multiplexed analysis technique for screening herpesviruses. US 20030143571 and Földes-Papp et al. (2004) disclose a method for simultaneous detection of a plurality of herpesviruses using microarray. Striebel et al. (2004) disclose studies concerning human herpesvirus diagnosis with DNA microarrays using dendrimers. In the publication of Boriskin et al (2004) microarrays were used in CNS infections to detect 13 different pathogens including several herpesviruses using long probes.

Although several methods for detecting various herpesviruses have been disclosed in the prior art, still new methods are needed by which the efficiency and speed of the detection of herpesviruses could be improved. The proper detection methods should tolerate some sequence variation and new strains to arise (Dolan et al, 2004; Dolan et al, 2006; Norberg et al. 2006; Tyler et al, 2007). Also only few methods make possible the simultaneous detection of several herpesviruses from the same biological sample. In particular, only some publications report of a method, which can make a distinction between HHV-6A and HHV-6B viruses.

Primers used to amplify some of the herpesviruses are known from U.S. Pat. No. 6,897,057, JP 6253900 and Vesanen et al. (1996) Yamamoto and Nakamura (2000), Akhtar et al. (1996), Van den Veyver et al. (1998), WO 9325707, US 20040110195 and Saffronetz et al. (2003). Promoter sequence for T3 RNA polymerase used in the amplification of specific nucleic acids is known from JP2001095590, EP 510085 and US 2004/0125774.

### SUMMARY

It is an aim of the present invention to provide a new and improved method for detecting herpesviruses.

In particular, it is an aim of the present invention to provide an efficient and rapid method for the detection of one or more herpesviruses from the same biological sample and/or from different biological samples. More specifically, it is an aim of the present invention to provide a method, which makes possible efficient and rapid detection of several herpesviruses simultaneously.

It is another aim of the invention to provide a microarray for efficient and rapid detection of herpesviruses.

These and other objects, together with the advantages thereof over known methods and microarrays are achieved by the present invention, as hereinafter described and claimed.

The method of this invention is based on microarray technology. According to the method several different herpesviruses can be detected simultaneously from the same and/or different biological sample. Also several samples can be studied at the same time by the method. It is also possible to determine the genotype of the viruses in the same reaction.

According to an embodiment of the invention the method of the invention comprises the following steps:

extracting DNA from a biological sample;

amplifying the extracted DNA;

translating the amplified DNA to ssRNA;

hybridizing ssRNAs to oligonucleotide sequences on a microarray plate, said oligonucleotide sequences corresponding to each of the herpesviruses to be detected;

extending the hybridised ssRNA by primer extension method in the presence of detectable nucleotides, and

detecting a signal from the detectable nucleotides by a suitable method.

More specifically, the method is characterized by what is stated in claims 1, and 23.

According to another embodiment of the invention a microarray for detecting herpesviruses comprises:

a solid support comprising at least one array; and

said array comprising oligonucleotides specific for at least one herpesvirus.

More specifically, the microarray is characterized by what is stated in claims 13 and a method for preparing a microarray for detecting herpesviruses is characterized by what is stated in claim 19.

A microarray kit is characterized by what is stated in claims 20 and an oligonucleotide is characterized by what is stated in claims 21.

A primer is characterized by what is stated in claim 22.

Diagnostic PCRs are commonly done for one herpesvirus at a time and this increase the detection time for several herpesviruses. Double and triple infections could be lost with PCRs whereas multiple infections may be detected by microarray in a day. The simultaneous detection of several herpesviruses is of great advantage. According to one preferred embodiment of the invention under suitable conditions the specificity of the microarray method of the present invention was found to be up to 100%. The microarray-based detection offers a fast and convenient protocol for detection of several herpesviruses for both diagnostics and research purposes. The method is in particular suitable for qualitative detection of herpesviruses.

### DESCRIPTION OF THE FIGURES

FIG. 1. Identification of eight herpesviruses by microarray-based method by using imaging. All the herpesvirus-specific oligonucleotides were spotted in 2×triplicate per array and unspecific oligonucleotides twice per array. Each array contained 60 spots (5×12 matrix). There were no signals observed from unspecific oligonucleotides. These images of HSV-1, HSV-2, CMV, EBV, HHV-7 and VZV were from average levels of viral DNA controls (range area 1000-5000 copies or VPs). In HHV-6A and -6B images no cross-reactions were observed in low levels of viral DNA (levels in image: 290 copies and 220 VPs, respectively).

### DETAILED DESCRIPTION OF THE INVENTION

“Herpesviruses” belong to family Herpesviridae. They are composed of a core of double-stranded DNA within an icosahedral capsid and a phopholipid bilayer.

Of the more than 100 known herpesviruses, eight infect humans. Human herpesviruses are classified into three subfamilies—alpha-, beta- and gamma-herpesviruses—on the basis of the length of viral replication cycle and host tissue range. Herpes simplex virus 1 (HSV-1), Herpes simplex virus 2 (HSV-2) and varicella-zoster virus (VZV) belong to subfamily alpha, cytomegalovirus (CMV), herpesvirus 6 (HHV-6) and herpesvirus 7 (HHV-7) belong to subfamily beta and Epstein-Barr virus (EBV) and herpesvirus (HHV-8) belong to subfamily gammaherpesviruses. Human infections are very common and patients can be infected with one or more herpesviruses, for example with both HSV-1 and HSV-2.

By the method of the present invention it is possible to detect one or more of a plurality of herpesviruses in a biological sample. Preferably the viruses are selected from the group comprising HSV-1, HSV-2, cytomegalo- (CMV), Epstein-Barr (EBV), varicella zoster (VZV), human herpes virus 6 (HHV-6A and HHV-6B) and human herpes virus 7 (HHV-7). In particular, by the method of the present invention can be made a distinction between HHV-6A and HHV-6B.

More specifically, by the method of the present infection it is possible to detect simultaneously at least two, preferable at least three, more preferably at least four different herpesviruses from the same sample.

Furthermore, the present invention allows successful detection of herpesviruses even when possible new strains arise. This is because several oligonucleotides per herpesvirus allow more sequence variation.

According to one preferred embodiment of the invention herpesviruses classified to the same subfamily, alpha-, beta- or gamma, are detected by the method of the present invention simultaneously.

According to another preferred embodiment of the invention herpesviruses commonly causing a double or triple infection in a patient are detected by the method of the present invention simultaneously.

Furthermore, other pathogens commonly causing infection at the same time as herpesviruses may be detected simultaneously.

By “biological sample” is here meant any sample from biological origin. In particular the sample is a clinical sample from a subject, preferably from a human subject. The sample may comprise whole blood, plasma, cerebrospinal fluid (CSF) sample, biopsy, urine, stool, sputum, mucus, bone marrow, skin, hair, nails, a lesional or ulcerous swab, a cervical/vaginal swab, a nasopharyngeal swab, a bronchoalveolar lavage, an endotracheal aspirate or any other clinical sample. Preferred clinical samples are plasma, whole blood and cerebrospinal fluid (CSF) samples.

The method of the present invention comprises that DNA is extracted from the biological sample to be studied. DNA can be extracted from the biological sample by using well-known DNA extraction methods. In the extraction can be used for example commercially available kits, such as MagNA Pure LC instrument and Total Nucleic Acid Kit (Roche Diagnostics, Basel, Switzerland), High Pure Viral Nucleic Acid Kit (Roche Diagnostics) or chloroform-phenol extraction.

The extracted DNA can then be amplified by using a suitable method. Preferably the amplification method is a PCR (polymerase chain reaction) method. PCR refers to the method for increasing the concentration of a segment of a target sequence in a mixture of DNA without cloning or purification. This process for amplifying the target sequence consists of introducing a large excess of two oligonucleotide primers to the DNA mixture containing the target sequence, followed by a precise sequence of thermal cycling in the presence of a DNA polymerase. The two primers are complementary to their respective strands of the double stranded target sequence. In this case the target sequence is a herpes virus sequence to be detected. It may also be a sequence of another pathogen to be detected. Because the desired amplified segments of the target sequence become the predominant sequences (in terms of concentration) in the mixture, they are said to be “PCR amplified”.

The primers for the PCR can be prepared according to methods well known in the art and described, e.g., in Sambrook, J. Fritsch, E. F., and Maniatis, T. (1989). Molecular Cloning: A Laboratory Manual, Cold Spring Harbor Laboratory Press, Cold Spring Harbor, N.Y.). The primer pair typically consists of a forward primer (sense) and reverse primer (antisense) corresponding to the specific areas of a herpesvirus sequence to be detected. The primer pair can be designed using commercially available software aimed for primer design, for example Primer3-software (http://frodo.wi.mit.edu/cgi-bin/primer3/primer3_www.cgi; Rozen and Skaletsky, 2000) and DNA mfold software (http://www.bioinfo.rpi.edu/applications/mfold/old/dna/; Zuker, 2003.

The amplification of the sample DNA can be made in one or several PCR amplification reactions. If more than one primer pair is used in the PCR, the method is called multiplex PCR. It is possible to amplify the DNA in one or more multiplex PCRs. For example here is disclosed the amplification of primer pairs for two or more herpesviruses in one multiplex PCR and primer pairs for two or more other herpesviruses in another multiplex PCR. If the DNA from one sample has been amplified in more than one PCR reaction, the PCR products from different reactions can be combined before further steps are carried out.

As is disclosed here a primer pair is designed for each herpesvirus to be detected. The primer pair comprises primer A and primer B, wherein advantageously

A is SEQ ID NO: 1 and B is SEQ ID NO: 3;

A is SEQ ID NO: 6 and B is SEQ ID NO: 7;

A is SEQ ID NO: 9 and B is SEQ ID NO: 10;

A is SEQ ID NO: 12 and B is SEQ ID NO: 13;

A is SEQ ID NO: 15 and B is SEQ ID NO: 16; or

A is SEQ ID NO: 19 and B is SEQ ID NO: 20.

According to a preferred embodiment of the invention SEQ ID NO: 22 can be used instead of SEQ ID NO: 1 and SEQ ID NO: 23 can be used instead of SEQ ID NO: 3. These sequences can be used also in addition to the original sequences.

Primer A is preferably a forward primer and primer B is preferably a reverse primer.

It should be understood that equivalent primer sequences comprise sequences, which have at least 90% identity, preferably at least 95%, more preferably at least 97% identity to the above mentioned primer pair sequences A or B. More preferably the sequences have at least 99% identity, most preferably 100% identity to the mentioned sequences. In particular there may be differences in the 5′ ends of the primers.

“Primer sequence having essentially the same sequence” refers to a primer sequence lacking one nucleotide, having one additional nucleotide, or having one change in the nucleotide sequence compared to the primer sequences SEQ ID NO: 1, SEQ ID NO: 3; SEQ ID NO: 6, SEQ ID NO: 7, SEQ ID NO: 9, SEQ ID NO:10, SEQ ID NO: 12, SEQ ID NO:13, SEQ ID NO: 15, SEQ ID NO:16, SEQ ID NO: 19, SEQ ID NO: 20, SEQ ID NO:22 or SEQ ID NO: 23. It may be also of advantage if there is nucleic acid variance, for example T/C, in primers (both primer alternatives available in the same mixture).

The primers as used herein refer to primers having a contiguous sequence of 10 to 50 nucleotides, preferably 20 to 40, more preferably 20 to 30 nucleotides. Primers used to perform the PCR reaction encompass nucleotide sequences of sufficient length and appropriate sequence so as to provide initiation of polymerization on a nucleic acid molecule.

In particular, primer pair SEQ ID NO: 1 and SEQ ID NO: 3 is designed for amplifying HSV-1 and HSV-2, primer pair SEQ ID NO: 6 and SEQ ID NO: 7 is designed for amplifying VZV, primer pair SEQ ID NO: 9 and SEQ ID NO: 10 is designed for amplifying CMV, primer pair SEQ ID NO: 12 and SEQ ID NO: 13 is designed for amplifying EBV, primer pair SEQ ID NO: 15 and SEQ ID NO:16 is designed for amplifying HHV-6, and primer pair SEQ ID NO: 19 and SEQ ID NO: 20 is designed for amplifying HHV-7. According to an alternative, preferred embodiment of the invention primer pair SEQ ID NO: 22 and SEQ ID NO: 23 is designed for amplifying HSV-1 and HSV-2.

The PCR amplification reaction is carried out under suitable conditions for the function of the DNA polymerase in the reaction mixture. The reaction mixture comprises DNA polymerase, all four nucleotide triphosphates (NTPs), a suitable buffer and optionally salts or other reagents. The amplification may consist for example of denaturation at about 95° C. for 10 min, followed by 40 cycles at 93-97° C., preferably 96° C. for 10 sec, 50-60° C. preferably 55° C. for 20 sec and about 69-74° C., preferably 72° C. for 20 sec and final extension 69-74° C., preferably 72° C. for 5 min.

Alternatively, the amplification may consist for example of denaturation at about 95° C. for 10 min, followed by 30 cycles at 93-97° C., preferably 96° C. for 10 sec, 45-70° C. preferably 65° C. for 20 sec (a 0.5° C. decrease per cycle) and about 69-74° C., preferably 72° C. for 20 sec, 10 cycles at 93-97° C., preferably 96° C. for 10 sec, 55-65° C. preferably 60° C. for 20 sec and final extension 69-74° C., preferably 72° C. for 5 min.

The length of the amplified DNA (amplicons) is preferably between 150 to 250 bp, more preferably 170 to 230 bp.

Once the sample DNA has been amplified the products are preferably converted to single stranded nucleic acid, preferably transcribed to single stranded RNA. In order to facilitate this step of the procedure one of the primers of the primer pair can include polymerase promoter of the polymerase used in the reaction. According to this disclosure the promoter of T3 RNA polymerase was included to the reverse primers. If a polymerase promoter is included to the primer, the length of the primer may be doubled. According to this disclosure the primer for the promoter of T3 RNA polymerase preferably comprises SEQ ID NO: 2. The transcribing reaction may be carried out by using commercially available kits, such as AmpliScribe™ T3 High Yield Transcription Kit (Epicentre, Madison, Wis.) or alternatively AmpliScribe™ T3-Flash™ Transcription Kit (Epicentre, Madison, Wis.).

Single stranded RNA is hybridised under suitable conditions to oligonucleotide sequences on a microarray plate, said oligonucleotide sequences corresponding to each of the herpesviruses to be detected.

As used herein “microarrays” refers to arrays comprising distinct oligonucleotides synthesized on a substrate, such as glass slide, silicon, plastic, a polymer matrix or any other suitable solid support. The microarray can be prepared and used according methods well-known for a person skilled in the art. The solid support may be e.g. a microscope glass slide or a silane-coated glass. The glass slide is preferably coated by isothiocyanate in order that aminated DNA-oligonucleotides can covalently bind to the glass (Lindroos et al. 2001).

Oligonucleotides for herpesviruses can be designed by using a suitable software, such as Primer3-software (http://frodo.wi.mit.edu/cgi-bin/primer3/primer3_www.cgi; Rozen and Skaletsky, 2000) and DNA mfold software (http://www.bioinfo.rpi.edu/applications/mfold/old/dna/; Zuker, 2003). The oligonucleotides are attached to the solid support by a suitable method. The oligonucleotides can e.g. be modified to comprise amino group at their 5′ end which can bind covalently to the silane—isothiocyanate layer on the solid support. The oligonucleotide may attach via its 5′ terminus to 3′-amino-propyltrimethoxysilane +1,4-phenylenedi-isothiocyanate-coated glass surface by formation of a covalent bond. The oligonucleotides comprise preferably also a poly (T) primer (T spacer) at their 5′ end before the specific oligonucleotide sequence. The length of the T spacer may be 6 to 12, preferably 8 to 10, typically 9 nucleotides. Also commercially available microarray glass slides having a suitable chemical surface, such as Asper Biotech, SAL-1 microarray slides, can be used.

The oligonucleotides as used herein refers to oligonucleotides having a contiguous sequence of about 10-50 nucleotides in length, preferably about 15-30, typically shorter than 30 nucleotides. More preferably the oligonucleotides are about 15-25 nucleotides in length, most preferably 16 to 23. Typically they may be about 20 nucleotides in length.

Oligonucleotides attached to the microarray encompass nucleotide sequences of sufficient length and appropriate sequence so as to provide hybridization of ssRNA of the tested herpes viruses in the sample.

Oligonucleotides may be spotted on the solid support by a suitable method, for example by a commercially available microarrayer such as OmniGrid®, GeneMachines, Huntingdon, UK. The array may consist of a matrix comprising the oligonucleotides for herpesviruses to be detected. The array may consist also of oligonucleotides with sequences unspecific for viruses as a control. In addition the array may consist also of oligonucleotides with sequences specific for other pathogens than herpesviruses. The solid support may contain 1 to 80, preferably 10 to 60 arrays. This means that on the same solid support can be studied simultaneously up to 80 samples.

The number of spots on an array is 4 to 200, preferably 10 to 100, more preferably 20 to 50, typically 30 to 40. The spots may consist of the oligonucleotides in duplicate or triplicate. They may consist of e.g. 10 to 25, typically 15-20 oligonucleotides in duplicate.

The amount of oligonucleotide per spot may be 0.5 to 1.5 pmol, typically about 1 pmol.

There may be about 200 spots per one array and the number of spots per one solid support, for example a slide may be about 16000.

The ssRNA is hybridised to the oligonucleotides and a primer extention reaction is carried out under suitable conditions. The hybridisation temperature may be 40 to 45° C., preferably about 42° C. The hybridisation time may be about 15 to 30 minutes. The hybridisation may be carried out in varying salt concentrations, for example in varying amounts of NaCl.

The primer extension reaction is carried out in the presence of a reverse transcriptase enzyme and nucleotide triphosphates, optionally with suitable buffers, salts and other reagents. The reaction may be carried out at the temperature of at least 42° C., preferably at the 49 to 55° C., more preferably at the temperature of about 52° C. The reaction may be carried out in 15 to 30 min, preferably about 20 min. Preferably the reaction mixture comprises detectable nucleotides, such as fluorescent nucleotides, the signal of which can be detected by a suitable method.

The detectable nucleotide may comprise any detectable reporter or signal moiety including, but not limited to radioisotopes, enzymes, antigens, antibodies, spectrophotometric reagents, chemiluminescent reagents, fluorescent and any other light producing chemicals.

The microarrays may be analysed by a suitable method, such as ScanArray Express scanner, ScanArray™ and QuantArray™ software (PerkinElmer, Wellesley, Mass.). The signal of the oligonucleotide spot (minus local background signal) may be compared to signals of unspecific oligonucleotides. The level or amount of signals is adjusted for positive and negative result. According to this disclosure signals of at least three times higher were considered positive.

The oligonucleotides preferably comprise a nucleotide sequence selected from the group of sequences:

SEQ ID NO: 4, SEQ ID NO: 5, SEQ ID NO: 8, SEQ ID NO: 11, SEQ ID NO: 14, SEQ ID NO: 17, SEQ ID NO: 18 and SEQ ID NO: 21.

According to a preferred embodiment of the invention SEQ ID NO: 24 can be used instead of sequence SEQ ID NO: 4, SEQ ID NO: 25 or general oligonucleotide sequences SEQ ID NO: 26 or SEQ ID NO: 27 or several of them can be used instead of sequence SEQ ID NO: 5 and sequences SEQ ID NO: 28 or SEQ ID NO: 29 or both of them or general oligonucleotide sequences SEQ ID NO:30 or SEQ ID NO:31 or any of the sequences can be used instead of sequence SEQ ID NO: 8. General oligonucleotide sequence SEQ ID NO: 30 can be used instead of sequence SEQ ID NO: 11, general oligonucleotide sequence SEQ ID NO:31 can be used instead of sequence SEQ ID NO: 14, and general oligonucleotide sequence SEQ ID NO:32 or any of the sequences SEQ ID NO: 17, SEQ ID NO: 18 or SEQ NO:32 can be used instead of sequences SEQ ID NO: 17 and SEQ ID NO: 18. General oligonucleotide sequence SEQ ID NO: 33 can be used instead of sequence SEQ ID NO: 21. The alternative oligonucleotide sequences can be used also in addition to (together with) the original oligonucleotide sequences.

It should be understood that equivalent oligonucleotide sequences comprise sequences, which have at least 90% identity, preferably at least 95%, more preferably at least 97 % identity to the sequences SEQ ID NO:4, SEQ ID NO: 5, SEQ ID NO:8, SEQ ID NO:11, SEQ ID NO: 14, SEQ ID NO: 17, SEQ ID NO: 18, SEQ ID NO: 21, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33.

More preferably the sequences have at least 99% identity, most preferably 100% identity to the mentioned sequences. If there are differences in the sequence, the differences may preferably be in the 5′ end of the oligonucleotide.

“Oligonucleotide sequence having essentially the same sequence” refers to an oligonucleotide sequence comprising a sequence lacking one nucleotide, having one additional nucleotide, or having one change in the nucleotide sequence compared to the sequences SEQ ID NO:4, SEQ ID NO: 5, SEQ ID NO:8, SEQ ID NO:11, SEQ ID NO: 14, SEQ ID NO: 17, SEQ ID NO: 18, SEQ ID NO: 21, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33.

Additional nucleotides may be added to either end of the above defined sequence of the oligonucleotide. However, this may result in secondary structures, which may cause less successful hybridisation. The oligonucleotides may be designed so that their melting temperature, Tm, is 49 to 54° C. Depending on the temperature, there should be a smaller or higher number of free nucleotides at the 5′ end of the oligonucleotide. According to this disclosure the 5′ end comprises a T spacer. The 5′ end of the oligonucleotide may comprise in addition to the T spacer 1 or 2 additional nucleotides. However, it is of advantage, if the 5′ end does not comprise any additional nucleotides. The 5′ end may also lack 1 or 2 nucleotides. However, it is also of advantage, if the 5′ end does not lack any nucleotide.

According to a preferred embodiment of the invention the microarray comprises oligonucleotides corresponding to at least two, preferably at least three different herpesviruses, more preferably at least four different herpesviruses. According to a preferred embodiment of the invention the microarray comprises oligonucleotides corresponding to 2 to 8 different herpesviruses. According to most preferred embodiments, the microarray comprises oligonucleotides corresponding to at least five, preferably at least six, more preferably at least seven or at least eight different herpesviruses. Only a few publications describe methods, by which the detection of seven or eight different herpes viruses is possible. Very few methods make possible the detection of HHV-6A and HHV-6B viruses. It is of particular advantage that all of the mentioned 8 herpesviruses can be detected simultaneously from the same biological sample.

According to one preferred embodiment of the invention at least oligonucleotides comprising sequences selected from the group consisting of SEQ ID NO:4 and SEQ ID NO:5 or at least oligonucleotides comprising sequences selected from the group consisting of SEQ ID NO:4, SEQ ID NO:5 and SEQ ID NO:8 are used to detect herpesviruses simultaneously.

Alternatively, at least oligonucleotides comprising sequences selected from the group consisting of SEQ ID NO:24, SEQ ID NO: NO:25, SEQ ID NO:26 and SEQ ID NO:27 or at least oligonucleotides comprising sequences selected from the group consisting of SEQ ID NO:24, SEQ ID NO: NO:25, SEQ ID NO:26, SEQ ID NO:27, SEQ ID NO:28, and SEQ ID NO:29.

According to another preferred embodiment of the invention at least oligonucleotides comprising sequences selected from the group consisting of SEQ ID NO: 11, SEQ ID NO: 14, SEQ ID NO: 17 and SEQ ID NO: 18, or at least oligonucleotides comprising sequences selected from the group consisting of SEQ ID NO: 11, SEQ ID NO: 14, SEQ ID NO: 17, SEQ ID NO: 18, SEQ ID NO 21, SEQ ID NO: 30, SEQ ID NO: 31, SEQ ID NO: 32 and SEQ ID NO: 33 are used to detect herpesviruses simultaneously.

According to one further preferred embodiment of the invention oligonucleotides comprising sequences detecting herpesviruses commonly causing a double or triple infection in a patient are detected by the method of the present invention simultaneously.

According to another further preferred embodiment of the invention the method comprises the detection of other pathogens or other viruses from the same biological sample. The other pathogens or viruses may be those commonly causing infection at the same time as herpesviruses. Alternatively, the other pathogens or viruses may be those causing central nervous system disorders or immune deficiency diseases. Examples of possible other pathogens are enteroviruses or borrelia causing pathogens.

The present invention provides also a microarray kit, which comprises the microarray and optionally reagents needed in the reaction. Such reagents are for example enzymes, nucleotide triphosphates (NTPs) and buffers.

By “qPCRs” is here meant quantitative PCR.

By “qualitative PCR” is meant here diagnostic PCR. The method of the present invention has been exemplified here by designating a microarray for the detection of eight herpesviruses. Microarray-based detection was compared to every-day diagnostic PCRs. Every-day diagnostic PCRs were exemplified by preparing two multiplex-PCRs.

The clinical specimens represented true patient cases. Concordant results from diagnostic PCRs and microarray were obtained in 89%. Microarray gave positive result in four cases whereas diagnostic PCRs remained negative.

There were two double infections (CMV-EBV) among CMV-positive plasma specimens detected by microarray. These specimens were from renal and bone marrow transplant patients. Both patients were seropositive for EBV and CMV. Among EBV-positive specimens we detected two double infections (EBV-CMV and EBV-HHV-7) with microarray. The EBV-CMV co-infection was a bone marrow transplanted patient, who was seropositive for EBV and had CMV IgM and IgG antibodies. In the case of EBV-HHV-7 infection the sample from a renal transplant patient was seronegative for HHV-7.

Two VZV-positive CSF samples were VZV-HHV-6B and VZV-EBV positive by microarray. Earlier studies had shown that the VZV-HHV-6B positive patient had also intrathecal antibodies against VZV and HHV-6. In the possible VZV-EBV co-infection, VZV IgG was verified in serum. One possible triple infection (HSV-2, HHV-7 and HHV-6B) was found by microarray. The sample was seronegative for HHV-7 and tested negative against HSV-1 and -2 by diagnostic PCR.

CSF cell counts, protein and glucose values were available for only 3 VZV-positive and 1 HSV-2-positive (possible triple infection) patients. In two of these, the findings were normal, whereas the other two showed elevated leukocyte and erythrocyte counts, normal glucose values and elevated protein levels. The HSV-2-positive patient had also HSV-IgM antibodies in CSF.

In diagnostics HHV-7 is not tested from clinical specimens by PCR. Four specimens, that were HHV-7 positive by microarray, were seronegative for HHV-7 at the time of sampling. Therefore, acute infections cannot be ruled out. HHV-7 is quite commonly found, e.g. in transplant patients (Dockrell et al, 2001).

Diagnostic PCRs are commonly done for one herpesvirus at a time and this increases the detection time for several herpesviruses. Double and triple infections could be lost with PCRs whereas multiple infections may be detected by microarray in a day. We conclude that microarray-based detection offers a fast and convenient protocol for detection of several herpesviruses for both diagnostics and research purposes.

The present invention increases the detection area of PCR amplicons. Several oligonucleotides per herpesvirus allow more sequence variation so that the detection succeeds even when the possible new strains arise. The coding sequence of herpesvirus DNA polymerase is a quite stable area but some point mutations might arise and the ssRNA hybridization to oligonucleotide and proper primer extension reaction might fail due the mutation. When the larger area of the sequence of PCR amplicon is detected with several oligonucleotides some of these oligonucleotides are working properly despite the possible mutation.

### EXAMPLES

### Example 1

### Viral DNA and Clinical Specimens

In setting up of multiplex-PCRs and microarray, serial 10-fold dilutions of commercial viral DNA controls (started from million copies or virus particles) and cell culture supernatants were used (Table I).

TABLE I
List of commercial viral DNA controls (Autogen Bioclear, Wiltshire,
UK) and cell supernatants used for optimization and testing of
multiplex-PCR and microarray.

| Virus | Strain | Quality of sample |
|---|---|---|
| HSV-1 | MacIntyre | commercial viral DNA |
| HSV-2 | G | |
| CMV | AD169 | |
| EBV | B95-8 | |
| HHV-7 | H7-4 | |
| HHV-6A | U1102 | |
| HHV-6B | Z-29 | |
| VZV | Rodstrain | |
| HHV-6A | house control* | cell supernatant |
| | GS | |
| HHV-6B | Z-29 | |

*Clinical isolate used as a house control.

In total, 116 clinical specimens were collected from pre-tested clinical material containing plasma (73), whole blood (10), cerebrospinal fluid (CSF) samples (23) and proficiency-testing samples (10) (QCMD, Glasgow, Scotland, UK). Plasma, whole blood and CSF samples were collected from solid organ and bone marrow transplant patients, and patients with neurological symptoms. Of the proficiency-testing samples, 4/10 were derived from a VZV and 6/10 from an HSV proficiency program 2004 (Table II). To study the specificity of the microarray, we tested 70 sera from patients (suspected epidemic nephropathy), 30 CSFs tested negative for HSV, EBV, CMV, VZV, HHV-6 and -7 DNA and 11 samples (Table II) from an enterovirus proficiency program 2004.

TABLE II
Virus proficiency programs, strains, viral DNA loads and panel formats
of proficiency testing samples used for testing of microarray.

| Virus | | | |
|---|---|---|---|
| proficiency | | Viral DNA | |
| program | Strain | load (GEq/ml) | Panel format |
| Varicella- | VZV strain 9/84 SMI | 700 | Lyophilized |
| zoster | | | cultured VZV |
| virus, | | | |
| 2004 | | | |
| | | 70 000 | |
| | | 7 000 000 | |
| | HSV-1 ATCC | 210 000 000 | |
| | strain MacIntyre | | |
| Herpes | HSV-1 ATCC | 24 000 | Freeze dried |
| simplex | strain MacIntyre | | inactived |
| virus, 2004 | | | samples |
| | HSV-2 ATCC strain MS | 1500 | |
| | | 15 000 | |
| | | 15 000 000 | |
| | VZV strain 9/84 SMI | 500 000 | |
| | neg | — | |
| Enterovirus, | selected EV serotypes | — | Lyophilized |
| 2004* | | | cultured EV |

GEq/ml, genome equivalent per ml;
neg, negative;
EV, enterovirus.
All proficiency testing samples were diluted in sterile water (1 ml).
*Eleven enteroviral proficiency testing samples contained enteroviruses (8/11), rhinoviruses (1/11) and 2 were negative.

### DNA Extraction

DNA was extracted using MagNA Pure LC instrument and Total Nucleic Acid Kit (Roche Diagnostics, Basel, Switzerland), High Pure Viral Nucleic Acid Kit (Roche Diagnostics) or chloroform-phenol extraction depending on the protocol used in diagnostics.

### Multiplex-PCR Primers and Oligonucleotides

The primer pair for HSV-1 and 2 (multiplex-PCR1) was modified from published primers (Piiparinen and Vaheri, 1991). Oligonucleotides and primers for CMV, EBV, VZV, HHV-6A, -6B and HHV-7 (multiplex-PCR2) were designed using Primer3-software (http://frodo.wi.mit.edu/cgi-bin/primer3/primer3_www.cgi; Rozen and Skaletsky, 2000) and DNA mfold software (http://www.bioinfo.rpi.edu/applications/mfold/old/dna/; Zuker, 2003). T3 RNA polymerase promoter sequence was included in the reverse multiplex-PCR primers (Tables III and IV).

Alternatively oligonucleotides and primers for HSV-1, HSV-2, CMV, EBV, VZV, HHV-6A, -6B and HHV-7 (multiplex-PCR1 and -2) were designed using Primer3-software (http://frodo.wi.mit.edu/cgi-bin/primer3/primer3_www.cgi; Rozen and Skaletsky, 2000) and DNA mfold software (http://www.bioinfo.rpi.edu/applications/mfold/old/dna/; Zuker, 2003).

Alternative sequences in Table III are SEQ ID NO: 22 to 26. Alternative sequences in Table IV are SEQ ID NO: 28 and 29.

TABLE III

Primers used for multiplex-PCR1 and oligonucleotides on microarray.

| Genus, | | | | |
|---|---|---|---|---|
| GenBank | | | | |
| accession | | | | |
| No. | Oligonucleotides | Gene name | Sequences (5′→3′) | Position |
| HSV-1, | HSV-FW | DNA pol., | AAGGAGGCGCCCAAGCGCCCG | 64750-64768 |
| X14112 | | UL30 | (SEQ ID NO: 1) | |
| | | | AGCGAATTCGAGATGCTG(T/C)T | 64130-64149 |
| | | | (SEQ ID NO: 22) | |
| | HSV-RV | | AATTAACCCTCACTAAAGGGAGA | |
| | | | (SEQ ID NO: 2) | |
| | | | TGGGGTACAGGCTGGCAAAGT | 64976-64956 |
| | | | (SEQ ID NO: 3) | |
| | | | CCTT(T/G)ATCTTGCTGCGCTTC | 64355-64336 |
| | | | (SEQ ID NO: 23) | |
| | HSV-1-T3 | | Am-TTTTTTTTTGTCCTTGACCCCACTT | 64907-64922 |
| | | | (spacer + | |
| | | | SEQ ID NO: 4) | |
| | | | Am-TTTTTTTTTCAAGCTGACGGACATT | 64237-64252 |
| | | | (spacer + | |
| | | | SEQ ID NO: 24) | |
| HSV-2, | HSV-FW | DNA pol., | AAGGAGGCGCCCAAGCGCCCG | 65209-65229 |
| Z86099 | | UL30 | (SEQ ID NO: 1) | |
| | | | AGCGAATTCGAGATGCTG(T/C)T | 64591-64610 |
| | | | (SEQ ID NO: 22) | |
| | HSV-RV | | AATTAACCCTCACTAAAGGGAGA | |
| | | | (SEQ ID NO: 2) | |
| | | | TGGGGTACAGGCTGGCAAAGT | 65449-65429 |
| | | | (SEQ ID NO: 3) | |
| | | | CCTT(T/G)ATCTTGCTGCGCTTC | 64816-64797 |
| | | | (SEQ ID NO: 23 | |
| | HSV-2-T3 | | Am-TTTTTTTTTAGGATAAGGACGACGAC | 65279-65295 |
| | | | (spacer + | |
| | | | SEQ ID NO: 5) | |
| | | | Am-TTTTTTTTTCAAGCTGACGGAGATC | 64698-64713 |
| | | | (spacer + | |
| | | | SEQ ID NO: 25) | |
| HSV (HSV- | HSV-GEN1-T3 | | Am-TTTTTTTTTGGTACAACATCATCAACTTC | HSV-1: 64197- |
| 1, X14112; | | | (spacer + | 64216; HSV-2: |
| HSV-2, | | | SEQ ID NO: 26) | 64658-64677 |
| Z86099) | | | | |
| | HSV-GEN2-T3 | | Am-TTTTTTTTTGGGACATAGGCCAGAG | HSV-1: 64311- |
| | | | (spacer + | 64326; HSV-2: |
| | | | SEQ ID NO: 27) | 64722-64787 |

FW, forward primer (sense); RV, reverse primer (anti-sense, T3 RNA polymerase promoter sequence AATTAACCCTCACTAAAGGGAGA before virus sequence); T3, oligonucleotide (sense, 9 × T spacer arm before sequence); Am, amino-link. Nucleic acid pair in parenthesis, such as for example Hsv-fw (T/C), means that there is nucleic acid variance in primers, i.e. there are both primer alternatives available in the same mixture. GEN, general (oligo for both HSV: s).

TABLE IV

Primers used for multiplex-PCR2 and oligonucleotides on microarray.

| Genus, | | | | |
|---|---|---|---|---|
| GenBank | | | | |
| accession No. | Oligonucleotides | Gene name | Sequences (5′→3′) | Position |
| VZV, | VZV-FW | DNA pol., | CCATTTTCTCGCCGATTTTA | 48508-48527 |
| AY548171 | | ORF28 | (SEQ ID NO: 6) | |
| | VZV-RV | | AATTAACCCTCACTAAAGGGAGA | |
| | | | (SEQ ID NO: 2) | |
| | | | GCCGCATTTGAACGTTTTAT | 48670-48651 |
| | | | (SEQ ID NO: 7) | |
| | VZV-T3 | | Am-TTTTTTTTTACCTCGTACGCTTTTTG | 48597-48613 |
| | | | (spacer + | |
| | | | SEQ ID NO: 8) | |
| | VZV1-T3 | | Am-TTTTTTTTTAGAATCCGTATCTCCATATA | 48569-48588 |
| | | | (spacer + | |
| | | | SEQ ID NO: 28 | |
| | VZV2-T3 | | Am-TTTTTTTTTCAGAATCCGTATCTCCATAT | 48568-48587 |
| | | | (spacer + | |
| | | | SEQ ID NO: 29 | |
| CMV, | CMV-FW | DNA pol., | GTACAACAGCGTGTCGTGCT | 57044-57063 |
| AY446894 | | UL44 | (SEQ ID NO: 9) | |
| | CMV-RV | | AATTAACCCTCACTAAAGGGAGA | |
| | | | (SEQ ID NO: 2) | |
| | | | CACCGGCCATCAAGTTTATC | 57240-57221 |
| | | | (SEQ ID NO: 10) | |
| | CMV-T3 | | Am-TTTTTTTTTGTAGAAGTTCTTCAGCTGC | 57122-57140 |
| | | | (spacer + | |
| | | | SEQ ID NO: 11) | |
| | GCMV-T3 | | Am-TTTTTTTTTGTTGATACGCATGTTTTT | 57143-57160 |
| | | | Spacer + | |
| | | | (SEQ ID NO: 30) | |
| EBV, | EBV-FW | DNA pol., | GGTAGATGACTCGAAGCTG | 154029-154047 |
| AJ507799 | | BALF5 | (SEQ ID NO: 12) | |
| | EBV-RV | | AATTAACCCTCACTAAAGGGAGA | |
| | | | (SEQ ID NO: 2) | |
| | | | ACCATCCTCGACAAGCAG | 154278-154261 |
| | | | (SEQ ID NO: 13) | |
| | EBV-T3 | | Am-TTTTTTTTTGAGAGGCAGGGAAAGAGG | 154184-154201 |
| | | | (spacer + | |
| | | | SEQ ID NO: 14) | |
| | GEBV-T3 | | Am-TTTTTTTTTGCACGTGCACTTGAT | 154237-154251 |
| | | | (spacer + | |
| | | | SEQ ID NO: 31) | |
| HHV-6A, | HHV-6-FW | DNA pol., | CTCGATCGAATCCGTAAACA | 59289-59308 |
| X83413 | | U38 | (SEQ ID NO: 15) | |
| | HHV-6-RV | | AATTAACCCTCACTAAAGGGAGA | 59444-59424 |
| | | | (SEQ ID NO: 2) | |
| | | | CCGCGTATGTTTTATCGAGAC | |
| | | | (SEQ ID NO: 16) | |
| | HHV-6A-T3 | | Am-TTTTTTTTTATCCTTGGACCGAGCTA | 59361-59377 |
| | | | (spacer + | |
| | | | SEQ ID NO: 17) | |
| HHV-6B, | HHV-6-FW | DNA pol., | CTCGATCGAATCCGTAAACA | 60409-60428 |
| AF157706 | | U38 | (SEQ ID NO: 15) | |
| | HHV-6-RV | | AATTAACCCTCACTAAAGGGAGA | |
| | | | (SEQ ID NO: 2) | |
| | | | CCGCGTATGTTTTATCGAGAC | 60564-60544 |
| | | | (SEQ ID NO: 16) | |
| | HHV-6B-T3 | | Am-TTTTTTTTTATCCTTGGACCGAACTC | 60481-60497 |
| | | | (spacer + | |
| | | | (SEQ ID NO: 18) | |
| HHV-6A, | GHHV-6-T3 | DNA pol., | Am-TTTTTTTTTCAGTAAATACTGTCGGTCTC | 59409-59428; |
| X83413 | | U38 | (spacer + | 60529-60548 |
| HHV-6B, | | | SEQ ID NO: 32) | |
| AF157706 | | | | |
| HHV-7, | HHV-7-FW | DNA pol., | AGGTCCAACATGCACAGTGA | 56787-56806 |
| AF037218 | | U38 | (SEQ ID NO: 19) | |
| | HHV-7-RV | | AATTAACCCTCACTAAAGGGAGA | |
| | | | (SEQ ID NO: 2) | |
| | | | GGCAAAGAAAATGTGGGCTA | 56993-56974 |
| | | | (SEQ ID NO: 20) | |
| | HHV-7-T3 | | Am-TTTTTTTTTGGATACAAACTTTGGAA | 56893-56909 |
| | | | (spacer + | |
| | | | (SEQ ID NO: 21) | |
| | GHHV-7-T3 | | Am-TTTTTTTTTCCAGGGTACTATAACACAGA | 56852-5687 |
| | | | (spacer + | |
| | | | SEQ ID NO: 33) | |

FW, forward primer (sense); RV, reverse primer (anti-sense, T3 RNA polymerase promoter sequence AATTAACCCTCACTAAAGGGAGA before virus sequence); T3, oligonucleotide (sense, 9 × T spacer arm before sequence); Am, amino-link.

### Multiplex-PCR Amplification

Multiplex-PCR1 was used to identify HSVs resulting in a 264-bp amplicon and in the alternative method 249-bp amplicon. Multiplex-PCR2 contained primer pairs for amplification of CMV, EBV, VZV, HHV-6A, -6B and -7 resulting in 220 bp, 273 bp, 186 bp, 179 bp (both HHV-6A and -6B) and 230 bp amplicons, respectively. Both multiplex-PCRs were carried out for each sample.

The multiplex-PCR1 was carried out in a final volume of 50 μl containing 50 mM KCl, 10 mM Tris-HCl (pH 8.3), 0.01% (w/vol) gelatine, 0.2 mM dNTPmix (Finnzymes, Espoo, Finland), 0.6 μM of primers, 12.5 U AmpliTaq Gold polymerase (Applied Biosystems, Foster City, Calif.) and 2 mM MgCl₂.

The multiplex-PCR2 was performed in a final volume of 53 μl containing 47.2 mM KCl, 9.4 mM Tris-HCI (pH 8.3), 0.009% (w/vol) gelatine, 0.19 mM dNTPmix (Finnzymes), 0.56 μM of each primers, 12.5 U AmpliTaq Gold polymerase (Applied Biosystems) and 1.9 mM MgCl₂. The amplification consisted of denaturation at 95° C. for 10 min, followed by 40 cycles at 96° C. for 10 sec, 55° C. for 20 sec and 72° C. for 20 sec and final extension 72° C. for 5 min.

### PCRs in Every-day Diagnostics

Two real-time quantitative PCRs (qPCRs) for CMV and EBV, and three qualitative PCRs for HSVs, HHV-6, and VZV were used in our every-day diagnostics. One of these PCRs and microarray-based method were performed in parallel for the clinical specimens using DNA from the same extraction and the results were compared qualitatively. The choice of the used diagnostic PCR was based on the primary diagnostic testing result and clinical information.

qPCRs for CMV and EBV were performed according to Piiparinen et al (2004) and a modification from Aalto et al (2003), respectively. In the latter, EBV primers, and probe designed by Kimura et al (1999) were used. The PCR for HSVs was performed according to Piiparinen et al (1991) using the probes reported by Vesanen et al (1996). PCR for HHV-6 was done using the primers designed by Gopal (1990) and the HHV-6 probe reported by Pitkaranta et al (2000). PCR for VZV was done according to Echevarria et al (1994) and Koskiniemi et al (1997). Microplate hybridization with luminometric detection (Vesanen et al, 1996) was used for the detection of qualitative PCR products.

### Preparation of Microarray

Microscope glass slides were activated as described by Guo et al (1994) with modification according to Pastinen et al (2000). The NH₂-modified oligonucleotides (Proligo, Paris, France) were spotted on slides using microarrayer (OmniGrid®, GeneMachines, Huntingdon, UK). The array consisted of a 5×12 matrix including eight oligonucleotides for herpesviruses (Table III and IV) and six oligonucleotides with sequences unspecific for viruses. The coated slides contained 50 arrays in which the herpesvirus-specific oligonucleotides were spotted in 2×triplicate and unspecific oligonucleotides functioning as negative hybridization controls twice per array. The spotting solution contained 20 μM oligonucleotide and 0.3 M sodium carbonate buffer (pH 9.0) or alternatively the commercial 1×microarray spotting solution (MSS, Arraylt, Telechem International, Sunnyvale, Calif.) in a final volume of 10 ρl. The spotting was performed at room temperature and 50% humidity. Slides were stored overnight at room temperature before use.

### In Vitro RNA Transcription and Microarray Reactions

Two multiplex-PCR products were pooled before transcribing into ssRNA using AmpliScribe™ T3 High Yield Transcription Kit (Epicentre, Madison, Wis.) or alternatively AmpliScribe™ T3 Flash Transcription Kit, 1 h 42° C.) following the manufacturer's instruction. Negative controls of extractions, multiplex-PCRs and PCRs were included.

Mini Pap Pen (Zymed, South Francisco, Calif.) was used to encircle the array area. The ssRNA solution containing 6 μI ssRNA and 1.5 mM NaCl in a final volume of 8.5 μl was heated at 95° C. for 1.5 min and allowed to react for 20 min at 42° C. with array. Alternatively the ssRNA 6 μl was heated at 96° C. for 2 min. After heating the ssRNA solution containing ssRNA 6 μl and 1.5 mM NaCl in a final volume of 8.5 μl was allowed to react for 20 min at 42° C. with array. After incubation the microarrays were washed with array washing buffer [0.5×TE (5 mM Tris, 0.5 mM EDTA), 0.3 M NaCl and 0.1% Triton X-100 (YA Kemia, Helsinki, Finland)] and sterile water.

The primer extension solution in a final volume of 4.5 μl containing 55 mM Tris-HCl, 11 mM MgCl₂, 83 mM KCl, 11 mM DTT (Epicentre), 0.6 μM dATP, dGTP, ddATP, ddGTP, dUTP-CY5 and dCTP-CY5 (Amersham Bioscience, Little Chalfont, UK), 5 U MMLV reverse transcriptase (Epicentre) and 0.5 M trehalose (Sigma-Aldrich) and 8.3% glycerol (MP Biomedicals, Irvine, Calif.) was added and allowed to react for 20 min at 52° C. Before scanning fluorescence, the microarrays were washed with array washing buffer and dried.

### Scanning and Analyzing the Microarray

The microarrays were analyzed using a ScanArray Express scanner, ScanArray™ and QuantArray™ software (PerkinElmer, Wellesley, Mass.). The analyzing cut-off value was determined for each array separately. The signal of the oligonucleotide spot (minus local background signal) were compared to signals of unspecific oligonucleotides. Signals at least three times more were considered positive. The microarray was carried out in duplicate. The number of quantified signals did not correlate to number of virus in specimens.

### Analytical Sensitivity

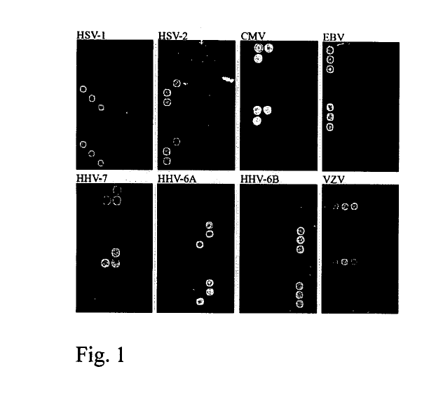

Multiplex-PCRs provided good analytical sensitivities (Table V) which were obtained by studying dilutions of commercial controls for each herpesvirus at minimum three times. Water controls of extractions and multiplex-PCRs were negative by microarray. Other cross-reactions than that between HHV-6A and -6B were not observed. At over 10000 HHV-6B copies/reaction cross-reactions between genotype-specific oligonucleotides were observed. In high concentration of HHV-6A, however, no cross-reactions were detected. Commercial controls worked properly in detection (FIG. 1) and furthermore showed the accuracy of the hybridization.

TABLE V
Sensitivities of multiplex-PCRs and microarray using commercial
viral DNA controls.

| | Sensitivities of | Sensitivity of |
|---|---|---|
| | multiplex-PCRs | microarray per |
| Virus | per reaction | reaction |
| HSV-1 | 9.1 VPs or | 9.1 VPs |
| | 30 copies* | 5 copies* |
| HSV-2 | 8.0 VPs or | 8.0 VPs or 5 copies* |
| | 30 copies* | |
| CMV | 3.5 copies | 1.0 copies |
| EBV | 10.0 copies | 3.0 copies |
| HHV-7 | 10.0 copies | 3.0 copies |
| HHV-6A | 29.0 copies | 7.0 copies |
| HHV-6B | 22.0 VPs | 2.5 VPs |
| VZV | 11.0 VPs or 10 | 4.5 VPs or 7.5 |
| | copies* | copies* |

VPs, virus particles. Sensitivities of multiplex-PCRs were defined by 2% gel electrophoresis which is less sensitive than hybridization methods.
*indicates result received by the alternative method, oligonucleotides and/or primers.

### Analysis of Clinical Specimens and Specificity

Altogether 116 specimens were tested simultaneously using one of the diagnostic PCRs and microarray. qPCR for CMV or EBV was performed for 73 plasma and qualitative HHV-6-PCR for 10 whole blood samples (Table VI). Qualitative HSV- or VZV-PCR was run for 23 CSFs (Table VII). Ten proficiency-testing specimens (Table VIII) were tested with qualitative HSV- and VZV-PCR. For 103 out of 116 (89%) clinical specimens, PCRs and microarray gave concordant qualitative results.

TABLE VI
Every-day diagnostic PCR and microarray results of plasma and
whole blood specimens.

| PCR result (number of | ¹Microarray result (number of |
|---|---|
| specimens) | specimens) |
| neg (32) | neg (28) |
| | CMV (1) |
| | EBV (1) |
| | HHV-6B (2) |
| CMV (26) | CMV (22) |
| | CMV and EBV (2) |
| | neg (2) |
| EBV (15) | EBV (12) |
| | EBV and CMV (1) |
| | EBV and HHV-7 (1) |
| | neg (1) |
| *HHV-6 (10) | HHV-6B (5) |
| | HHV-6B and EBV (2) |
| | HHV-6B and HHV-7 (2) |
| | neg (1) |

neg, negative. Specimens were stored at −70° C.
*whole blood specimens
¹Multiplex-PCRs detected by microarray.

Out of 32 PCR-negative plasma specimens, microarray was negative in 28 (Table VI). Four originally negative plasma specimens showed positive results with microarray. In 24/26 CMV-positive plasma specimens, qPCR and microarray gave concordant results. Two CMV-qPCR-positive samples, 1280 and 810 genome equivalents (GEq) per ml, remained negative by microarray. Two double infections of CMV and EBV were observed among CMV-positive cases (5420 GEq/ml for CMV and 6280 for EBV, and 4200 for CMV and 4130 EBV, respectively).

EBV was detected by microarray in all but one of 15 EBV-PCR-positive samples. The missed specimen gave 860 GEq/ml in qPCR. Two double infections (EBV-CMV and EBV-HHV-7) were detected with microarray. The specimens had 15×10⁶ and 990 GEq/ml for EBV in qPCR, respectively.

For 9 out of 10 HHV-6-PCR-positive specimens, HHV-6B was detected by microarray. Microarray detected also four double infections; two were HHV-6B-EBV, and the other HHV-6B-HHV-7 infections.

Altogether 10/23 CSF samples were negative and 13/23 positive by PCR. All PCR-negative CSFs were negative in microarray. Of 3 HSV-positive specimens microarray missed one HSV-2 positive sample. One triple infection (HSV-2, HHV-7 and HHV-6B) was observed by microarray. Both HHV-6-positive samples remained negative, whereas all 8 VZV-positive samples were positive also by microarray (Table VII).

TABLE VII
Every-day diagnostic PCR and microarray results of CSF specimens*.

| PCR result (number of | Microarray result (number of |
|---|---|
| specimens) | specimens) |
| neg (10) | neg (10) |
| HHV-6 (2) | neg (2) |
| HSV-1 (1) | HSV-1 (1) |
| HSV-2 (2) | HSV-2, HHV-7 and HHV-6B (1) |
| | neg (1) |
| VZV (8) | VZV (6) |
| | VZV and HHV-6B (1) |
| | VZV and EBV (1) |

neg, negative. Specimens stored at −70° C.

Two out of 10 proficiency-testing specimens were negative by PCR and microarray. Of eight PCR-positive specimens, two were positive for HSV-1, two for HSV-2, and four for VZV, as was previously been informed by the QCMD. Microarray remained negative in one of the HSV-1 and -2-PCR-positives, otherwise the results were concordant. In one VZV-positive sample a very weak HHV-6B microarray signal was seen in addition to VZV signal (Table VII).

The herpesvirus-negative panel (70 serum, 30 CSF and 11 proficiency-testing samples) was negative in multiplex-PCRs and microarray. Thus the specificity turned out to be 100% (results not included in the tables).

TABLE VIII
Every-day diagnostic PCR and microarray results of QCMD specimens.

| PCR result (number of | Microarray result (number of |
|---|---|
| specimens) | specimens) |
| neg (2) | neg (2) |
| HSV-1 (2) | HSV-1 (1) |
| | neg (1) |
| HSV-2 (2) | HSV-2 (1) |
| | neg (1) |
| VZV (4) | VZV (3) |
| | VZV and HHV-6B (1) |

neg, negative

### REFERENCES

Aalto S M, Juvonen E, Tarkkanen J, Volin L, Ruutu T, Mattila P S, Piiparinen H, Knuutila S, Hedman K. Lymphoproliferative disease after allogeneic stem cell transplantation—pre-emptive diagnosis by quantification of Epstein-Barr virus DNA in serum. J. Clin. Virol. 2003; 28:275-83.

Aberle S W, Puchhammer-Stockl E. Diagnosis of herpesvirus infections of the central nervous system. J. Clin. Virol. 2002; 1:S79-85.

Akhtar et al. PCR diagnosis of viral pneumonitis from fixed-lung tissue in children. Biochemical and Molecular Medicine 1996; 58:66-76.

Aurelius E, Johansson B, Sköldenberg B, Staland A, Forsgren M. Rapid diagnosis of herpes simplex encephalitis by nested polymerase chain reaction assay of cerebrospinal fluid. Lancet 1991; 337:189-92.

Boriskin Y S, Rice P S, Stabler R A, Hinds J, Al-Ghusein H, Vass K, Butcher P D. DNA microarrays for virus detection in cases of central nervous system infection. J. Clin. Microbiol. 2004; 42:5811-8.

Borsting C, Sanchez J J, Morling N. Multiplex PCR, amplicon size and hybridization efficiency on the NanoChip electronic microarray. Int. J. Legal Med. 2004; 118:75-82.

Boutolleau D, Fernandez C, Andre E, Imbert-Marcille B M, Milpied N, Agut H, Gautheret-Dejean A. Human herpesvirus (HHV)-6 and HHV-7: two closely related viruses with different infection profiles in stem cell transplantation recipients. J. Infect. Dis. 2003; 187:179-86.

Chiu S S, Cheung C Y, Tse C Y, Peiris M. Early diagnosis of primary human herpesvirus 6 infection in childhood: serology, polymerase chain reaction, and virus load. J. Infect. Dis. 1998; 178:1250-6.

Chizhikov V, Wagner M, Ivshina A, Hoshino Y, Kapikian A Z, Chumakov K. Detection and genotyping of human group A rotaviruses by oligonucleotide microarray hybridization. J. Clin. Microbiol. 2002; 40:2398-407.

Druce J, Catton M, Chibo D, Minerds K, Tyssen D, Kostecki R, Maskill B, Leong-Shaw W, Gerrard M, Birch C. Utility of a multiplex PCR assay for detecting herpesvirus DNA in clinical samples. J. Clin. Microbiol. 2002; 40:1728-32.

Dockrell D H, Paya C V. Human herpesvirus-6 and -7 in transplantation. Rev. Med. Virol. 2001; 11:23-36.

Echevarria J M, Casas I, Tenorio A, de Ory F, Martinez-Martin P. Detection of varicella-zoster virus-specific DNA sequences in cerebrospinal fluid from patients with acute aseptic meningitis and no cutaneous lesions. J. Med. Virol. 1994; 43:331-5.

Espy M J, Teo R, Ross T K, Svien K A, Wold A D, Uhl J R, Smith T F. Diagnosis of varicella-zoster virus infections in the clinical laboratory by LightCycler PCR. J. Clin. Microbiol. 2000; 38:3187-9.

Földes-Papp Z, Egerer R, Birch-Hirschfeld E, Striebel H-M, Demel U, Tolz G P and Wutzler P. Detection of multiple human herpes viruses by DNA microarray technology. Mol Diagn 2004; 8(1):1-9.

Gemignani F, Landi S, Chabrier A, Smet A, Zehbe I, Canzian F, Tommasino M. Generation of a DNA microarray for determination of E6 natural variants of human papillomavirus type 16. J. Virol. Methods 2004; 119:95-102.

Gopal M R, Thomson B J, Fox J, Tedder R S, Honess R W. Detection by PCR of HHV-6 and EBV DNA in blood and oropharynx of healthy adults and HIV-seropositives. Lancet 1990; 335:1598-9.

Guo Z, Guilfoyle R A, Thiel A J, Wang R, Smith L M. Direct fluorescence analysis of genetic polymorphisms by hybridization with oligonucleotide arrays on glass supports. Nucleic Acids Res. 1994; 22:5456-65.

Hermouet S, Sutton C A, Rose T M, Greenblatt R J, Corre I, Garand R, Neves A M, Bataille R, Casey J W. Qualitative and quantitative analysis of human herpesviruses in chronic and acute B cell lymphocytic leukemia and in multiple myeloma. Leukemia 2003; 17:185-95.

Huber M, Mundlein A, Dornstauder E, Schneeberger C, Tempfer C B, Mueller M W, Schmidt W M. Accessing single nucleotide polymorphisms in genomic DNA by direct multiplex polymerase chain reaction amplification on oligonucleotide microarrays. Anal. Biochem. 2002; 303:25-33.

Hudnall S D, Chen T, Tyring S K. Species identification of all eight human herpesviruses with a single nested PCR assay. J. Virol. Methods 2004; 116:19-26.

Ihira M, Yoshikawa T, Ishii J, Nomura M, Hishida H, Ohashi M, Enomoto Y, Suga S, Iida K, Saito Y, Nishiyama Y, Asano Y. Serological examination of human herpesvirus 6 and 7 in patients with coronary artery disease. J. Med. Virol. 2002; 67:534-7.

Kimura H, Morita M, Yabuta Y, Kuzushima K, Kato K, Kojima S, Matsuyama T, Morishima T. Quantitative analysis of Epstein-Barr virus load by using a real-time PCR assay. J. Clin. Microbiol. 1999; 37:132-6.

Koskiniemi M, Mannonen L, Kallio A, Vaheri A. Luminometric microplate hybridization for detection of varicella-zoster virus PCR product from cerebrospinal fluid. J. Virol. Methods 1997; 63:71-9.

Koskiniemi M, Piiparinen H, Rantalaiho T, Eränko P, Färkkilä M, Räihä K, Salonen E M, Ukkonen P, Vaheri A. Acute central nervous system complications in varicella zoster virus infections. J. Clin. Virol. 2002; 25:293-301.

Lindroos K, Liljedahl U, Raitio M, Syvanen AC. Minisequencing on oligonucleotide microarrays: comparison of immobilisation chemistries. Nucleic Acids Res. 2001 Jul 1; 29(13):E69-9.

Nordberg P, Liljeqvist J A, Bergstrom T, Sammons S, Smid D C, Loparev V N. Complete-genome phylogenetic approach to varicella-zoster virus evolutio: genetic idvergence and evidence for recombination. J. Virol 2006: 80: 9569-9576.

Pastinen T, Raitio M, Lindroos K, Tainola P, Peltonen L, Syvänen A C. A system for specific, high-throughput genotyping by allele-specific primer extension on microarrays. Genome Res. 2000; 10:1031-42.

Piiparinen H, Vaheri A. Genotyping of herpes simplex viruses by polymerase chain reaction. Arch. Virol. 1991; 119:275-283.

Piiparinen H, Höckerstedt K, Lappalainen M, Suni J, Lautenschlager I. Monitoring of viral load by quantitative plasma PCR during active cytomegalovirus infection of individual liver transplant patients. J. Clin. Microbiol. 2002; 40:2945-52.

Piiparinen H, Höckerstedt K, Grönhagen-Riska C, Lautenschlager I. Comparison of two quantitative CMV PCR tests, Cobas Amplicor CMV Monitor and TaqMan assay, and pp65-antigenemia assay in the determination of viral loads from peripheral blood of organ transplant patients. J. Clin. Virol. 2004; 30:258-66.

Pitkäranta A, Piiparinen H, Mannonen L, Vesaluoma M, Vaheri A. Detection of human herpesvirus 6 and varicella-zoster virus in tear fluid of patients with Bell's palsy by PCR. J. Clin. Microbiol. 2000; 38:2753-5.

Read S J, Kurtz J B. Laboratory diagnosis of common viral infections of the central nervous system by using a single multiplex PCR screening assay. J. Clin. Microbiol. 1999; 37:1352-5.

Read S J, Mitchell J L, Fink C G. LightCycler multiplex PCR for the laboratory diagnosis of common viral infections of the central nervous system. J. Clin. Microbiol. 2001; 39:3056-9.

Rozen S, Skaletsky H. Primer3 on the WWW for general users and for biologist programmers. Methods Mol. Biol. 2000; 132:365-86.

Safronetz D, Humar, A, Tipples, G A. Differentation and quantitation of human herpesviruses 6A, 6B, and by real-time PCR. Journal of Virological Methods 2003, 112:99-105.

Schirm J, J. J. Meulenberg J J, Pastoor G W, van Voorst Vader P C, Schroder F P. Rapid detection of varicella-zoster virus in clinical specimens using monoclonal antibodies on shell vials and smears. J. Med. Virol. 1989; 28:1-6.

Sengupta S, Onodera K, Lai A, Melcher U. Molecular detection and identification of influenza viruses by oligonucleotide microarray hybridization. J. Clin. Microbiol. 2003; 41:4542-50.

Striebel H-M, Birch-Hirschfeld E, Egerer R, Földes-Papp Z, Tilz G P, Stelzner A. Enhancing sensitivity of human herpes virus diagnosis with DANN microarrays using dendrimers 2004; 77:89-97

Suga S, Yoshikawa T, Nagai T, Asano Y. Clinical features and virological findings in children with primary human herpesvirus 7 infection. Pediatrics 1997; 99:E4.

Tanaka K, Kondo T, Torigoe S, Okada S, Mukai T, Yamanishi K. 1994. Human herpesvirus 7: another causal agent for roseola (exanthem subitum). J. Pediatr. 1994; 125:1-5.

Tyler S D, Peters G A, Grose C, Severini A, Gray M J, Upton C, Tipples G A. Genomic cartography of varicella-zoster virus: a complete genome-based analysis of strain variability with implications for attenuation and phenotypic differences. Virology 2007: 359:447-458.

Van den Veyver et al. Detection of intrauterine viral infection using the polymerase chain reaction. Molecular Genetics and Metabolism 1998; 63:85-95.

Vesanen M, Piiparinen H, Kallio A, Vaheri A. Detection of herpes simplex virus DNA in cerebrospinal fluid samples using the polymerase chain reaction and microplate hybridization. J. Virol. Methods 1996; 59:1-11.

Volokhov D, Chizhikov V, Chumakov K, Rasooly A. Microarray-based identification of thermophilic Campylobacter jejuni, C. coli, C. lari, and C. upsaliensis. J. Clin. Microbiol. 2003; 41:4071-80.

Wang D, Coscoy L, Zylberberg M, Avila P C, Boushey H A, Ganem D, DeRisi J L. Microarray-based detection and genotyping of viral pathogens. Proc. Natl. Acad. Sci. USA 2002; 99:15687-92.

Zuker M. Mfold web server for nucleic acid folding and hybridization prediction. Nucleic Acids Res. 2003; 13:3406-3415.

Yamamoto T, Nakamura, Y. A single tube PCR assay for simultaneous amplification of HSV-1/-2, VZV, CMV, HHV-6A/-6B, and EBV DNAs in cerebrospinal fluid from patients with virus-related neurological diseases. Journal of Neuro Virology 2000; 6:410-417.

What is claimed is:

1. A method for detecting one or more herpesviruses in a biological sample, said method comprising the steps of:
extracting DNA from the biological sample;
amplifying the extracted DNA;
translating the amplified DNA to ssRNA;
hybridizing the ssRNAs to oligonucleotide sequences on a microarray, said oligonucleotide sequences corresponding to each of the herpesviruses to be detected;
extending the hybridised ssRNA by primer extension method in the presence of detectable nucleotides;
detecting a signal from the detectable nucleotides by a suitable method;
wherein the length of the oligonucleotide sequence is less than 50 nucleotides and the sequence comprises a sequence in 5′ to 3′ direction selected from the group consisting of SEQ ID NO:4, SEQ ID NO:5, SEQ ID NO:8, SEQ ID NO:11, SEQ ID NO: 14, SEQ ID NO: 17, SEQ ID NO: 18, SEQ ID NO: 21, SEQ ID NO:24, SEQ ID NO: NO:25, SEQ ID NO: 26, SEQ ID NO: 27, SEQ ID NO:28, SEQ ID NO: 29, SEQ ID NO: 30, SEQ ID NO: 31, SEQ ID NO: 32, and SEQ ID NO:33 or a sequence having essentially the same sequence as any of the mentioned sequences.

2. The method according to claim 1, wherein the microarray comprises oligonucleotides corresponding to at least two different herpesviruses.

3. The method according to claim 1, wherein the microarray comprises oligonucleotides corresponding to at least three different herpesviruses.

4. The method according to claim 1, wherein the microarray comprises oligonucleotides corresponding to herpesviruses selected from the group consisting of
HSV-1, HSV-2, CMV, EBV, VZV, HHV6, HHV-6A, HHV-6B and HHV-7.

5. The method according to claim 1, wherein the detection of different herpesviruses is simultaneous.

6. The method according to claim 1, wherein the method further comprises detection of other pathogens, preferably viruses.

7. The method according to claim 1, wherein the method further comprises detection of diseases of the central nervous system (CNS) or immunodeficiency diseases.

8. The method according to claim 6, wherein the method further comprises detection of enteroviruses or borrelia.

9. The method according to claim 6, wherein the detection of different herpesviruses and other pathogens is simultaneous.

10. The method according to claim 1, wherein the amplification of the DNA is carried out by using a pair of primers comprising primer A and primer B selected from the group consisting of
A is SEQ ID NO: 1 and B is SEQ ID NO: 3;
A is SEQ ID NO: 6 and B is SEQ ID NO: 7;
A is SEQ ID NO: 9 and B is SEQ ID NO: 10;
A is SEQ ID NO: 12 and B is SEQ ID NO: 13;
A is SEQ ID NO: 15 and B is SEQ ID NO: 16;
A is SEQ ID NO: 19 and B is SEQ ID NO: 20; and
A is SEQ ID NO: 22 and B is SEQ ID NO: 23,
or a sequence having at least 90% identity with the mentioned sequences.

11. The method according to claim 1, wherein the amplification of the DNA is carried out by multiplex PCR in two reactions.

12. The method according to claim 11, wherein HSV-1 and HSV-2 are amplified in one reaction and CMV, EBV, VZV, HHV-6, HHV-6A, HHV-6B and HHV-7 are amplified in another reaction.

13. A microarray for detecting herpesviruses, said microarray comprising:
a solid support comprising at least one array;
said array comprising oligonucleotides specific for at least one herpesvirus, wherein the length of the oligonucleotide sequence is less than 50 nucleotides and the sequence comprises a sequence in 5′ to 3′ direction selected from the group consisting of SEQ ID NO:4, SEQ ID NO: 5, SEQ ID NO:8, SEQ ID NO:11, SEQ ID NO: 14, SEQ ID NO: 17, SEQ ID NO: 18 and SEQ ID NO: 21, SEQ ID NO:24, SEQ ID NO: NO: 25, SEQ ID NO: 26, SEQ ID NO: 27, SEQ ID NO:28, SEQ ID NO: 29, SEQ ID NO: 30, SEQ ID NO: 31, SEQ ID NO: 32, and SEQ ID NO:33 or a sequence having essentially the same sequence as any of the mentioned sequences.

14. The microarray according to claim 13, wherein the number of arrays on the solid support is 1 to 80, preferably 10 to 60.

15. The microarray according to claim 14, wherein the number of oligonucleotide spots on a microarray is 4 to 200, preferably 10 to 100.

16. The microarray according to claim 14, wherein the microarray is used for detecting a herpesvirus selected from the group comprising HSV-1, HSV-2, CMV, EBV, VZV, HHV-6, HHV-6A, HHV-6B and HHV-7.

17. The microarray according to claim 14, wherein the microarray comprises oligonucleotides for detecting further diseases as those caused by herpesvirus.

18. The microarray according to claim 13, wherein the microarray comprises oligonucleotides for detecting other pathogens, such as enteroviruses or borrelia causing pathogens

19. A method for preparing a microarray for detecting herpesviruses, said method comprising the steps of:
attaching oligonucleotides corresponding to at least one herpesvirus in an array;
multiplying the arrays onto a slide, the number of the arrays corresponding to the number of samples to be analysed.
wherein the length of the oligonucleotide sequence is less than 50 nucleotides and the sequence comprises a sequence in 5′ to 3′ direction selected from the group consisting of g SEQ ID NO:4, SEQ ID NO: 5, SEQ ID NO:8, SEQ ID NO:11, SEQ ID NO: 14, SEQ ID NO: 17, SEQ ID NO: 18 and SEQ ID NO: 21, SEQ ID NO:24, SEQ ID NO: NO: 25, SEQ ID NO: 26, SEQ ID NO: 27, SEQ ID NO:28, SEQ ID NO: 29, SEQ ID NO: 30, SEQ ID NO: 31, SEQ ID NO: 32, and SEQ ID NO:33 or a sequence having essentially the same sequence as any of the mentioned sequences.

20. A kit which comprises the microarray according to claim 13 and optionally, primers, buffers, enzymes and/or nucleotides.

21. An oligonucleotide comprising a sequence selected from the group consisting of: SEQ ID NO:4, SEQ ID NO: 5, SEQ ID NO:8, SEQ ID NO:11, SEQ ID NO: 14, SEQ ID NO: 17, SEQ ID NO: 18 and SEQ ID NO: 21, SEQ ID NO:24, SEQ ID NO: NO:25, SEQ ID NO: 26, SEQ ID NO: 27, SEQ ID NO:28, SEQ ID NO: 29, SEQ ID NO: 30, SEQ ID NO: 31, SEQ ID NO: 32, and SEQ ID NO:33 wherein the length of the oligonucleotide sequence is less than 50 nucleotides and the sequence comprises the mentioned sequence in 5′ to 3′ direction.

22. Primer selected from the group consisting of SEQ ID NO: 1, SEQ ID NO: 3, SEQ ID NO: 6, SEQ ID NO: 7, SEQ ID NO: 9, SEQ ID NO:10, SEQ ID NO: 12, SEQ ID NO:13, SEQ ID NO: 15, SEQ ID NO:16, SEQ ID NO: 19, SEQ ID NO: 20, SEQ ID NO: 22 and SEQ ID NO: 23 or a sequence having at least 90% identity with the mentioned sequences.

23. A method for detecting at least two herpes viruses simultaneously in a biological sample, said method comprising the steps of:
extracting DNA from a biological sample;
amplifying the extracted DNA;
translating the amplified DNA to ssRNA;
hybridizing the ssRNAs to oligonucleotide sequences on a microarray plate, said oligonucleotide sequences corresponding to herpesviruses commonly causing simultaneous infections;
extending the hybridised ssRNA by primer extension method in the presence of detectable nucleotides; and
detecting a signal from the detectable nucleotides by a suitable method.
